(19) United States
(12) Patent Application Publication
(10) Pub. No.: US 2013/0303999 A1
(43) Pub. Date: Nov. 14, 2013

(54) Needle Cannula with a Collapse Zone

(71) Applicants: Mikael Andersen, Malmoe (SE); Steffen Lav, Broenshoej (DK)

(72) Inventors: Mikael Andersen, Malmoe (SE); Steffen Lav, Broenshoej (DK)

(73) Assignee: NOVO NORDISK A/S, Bagsvaerd (DK)

(21) Appl. No.: 13/993,855

(22) PCT Filed: Dec. 5, 2011
(86) PCT No.: PCT/EP2011/071740

§ 371 Date: Aug. 1, 2013

Related U.S. Application Data
(60) Provisional application No. 61/425,363, filed on Dec. 21, 2010.

(30) Foreign Application Priority Data
Dec. 16, 2010 (EP) 10195375.0

(51) Int. Cl. A61M 5/32; G01B 5/12

(52) U.S. Cl. CPC A61M 5/32; G01B 5/12
USPC 604/273; 216/100; 73/37.9

(57) ABSTRACT

A metallic cylindrical tubular needle cannula (1) is subjected to a metal etching liquid inside the lumen (7) thereby increasing the inside diameter and enhancing the flow properties while maintaining the outside appearance. The inside wall (5) is further provided with a collapse zone (10) manufactured by radiating heat (11) to a specific outside area of the needle cannula (1) thereby increasing the aggression of the etchant. Further two different methods of in- process testing of the inside diameter of the needle cannulae using any fluid gas are disclosed.

### THE TECHNICAL FIELD OF THE INVENTION

The present invention relates to a needle cannula having an internal collapse zone in which zone the needle cannula is more bendable. The present invention further relates to a method for producing such needle cannula and to a general method for testing the internal diameter of any needle cannula.

### DESCRIPTION OF RELATED ART

Within the area of injection needles it is known that repeated bending of a needle cannula may cause the needle cannula to break. Bending e.g. occurs if a patient accidently drops the syringe with the injection needle mounted on to a floor or the like. The patient will most often try to straighten out the bended needle cannula rather than mounting a new injection needle on the syringe

Straightening a bended needle cannula typically induces strains in the needle cannula whereby microscopic cracks are formed in the surface of the needle cannula. These cracks propagate rapidly when the needle cannula is exposed to additional strain and the risk of breaking the needle cannula and in worst case leave the broken-of part of the needle cannula in the tissue of a patient is significantly increased.

Also, since the development of needle cannulae points in the direction of even thinner needle cannulae having extremely thin walls, microscopic cracks in the surface of the needle cannula become more critical.

Attempts have been made to heat-treat the walls of needle cannulae in order to provide an area with reduced hardness thereby making the needle cannula more sustainable to bending in that specific area.

U.S. Pat. No. 6,422,865 discloses a needle cannula having an annealed flexible distal portion which is easy bendable.

WO 2006/136364 discloses a needle cannula in which a region of the needle cannula has been exposed to heat in order to form a more ductile region, which region can be continuously bended.

Further it is known to decrease the wall thickness of needle cannulae by washing the inside surface of the cannula lumen with an etching reagent. This is e.g. disclosed in U.S. Pat. No. 3,326,785 and in PCT/EP2010/063787.

U.S. Pat. No. 3,326,786 disclose how a liquid electrolyte solution is pumped into a pressure chamber holding a single metallic needle cannula through which the solution flow.

PCT/EP2010/063787 discloses a process of transferring a pressurized metal etching liquid into only a part of the inside lumen of a number of needle cannulae.

### DESCRIPTION OF THE INVENTION

It is an object of the present invention to provide a needle cannula having enhanced safety against breakage. Following this it is an object of the present invention to provide a needle cannula which will collapse when bended such that it is literally impossible to straighten out the needle cannula and use the once bended needle cannula. The enhanced safety lies in the lack of opportunity to bend the needle cannula repeatedly.

Further, it is an object to provide a method for producing a needle cannula having a collapse zone. A further object is to provide a more general method for testing the size of the inside diameter of any needle cannula.

Claim 1

The optimal safety against breakage following bending of the needle cannula is to provide a needle cannula which can not be straighten out following bending.

Such needle cannula is provided by introducing a collapse zone internally in the needle cannula such that the needle cannula will always bend at the collapse zone but bend in a controlled manner where after the needle cannula can not be straighten out again.

By having the collapse zone provided in the inside surface it is also hidden from the user such that the outside visual appearance of the needle cannula is that of an ordinary needle cannula. During injection the outside surface of the needle cannula contacting the skin of the user is also that of any ordinary needle cannula.

Claim 2-5

The collapse zone is advantageously formed by removing a part of the material forming the wall of the needle cannula. By locally carving out a part of the material a region with a thinner wall thickness can be created. Such region will then collapse when the needle cannula is bended. The region carved out can have any physical shape, however, the shape of a cylinder, a circular cone or a spherical cap being perpendicular to the length of the needle cannula is preferred.

Claim 6-7

The internal diameter of the lumen is advantageously increased by flushing a metal etching liquid through the lumen of the needle cannula. Such etchant can be circulated through the entire length of the needle cannula or only through a part of the lumen as described in the prior art.

By heating a local area of the needle cannula the etchant will conduct a more aggressive behaviour in the heated area. If heat is applied to a restricted area, the etchant will in that specific area remove more material than in areas not heated.

If therefore heat is applied to e.g. a circular area of the outside surface of the needle cannula, material will be carved out in the inside surface subjected to the etchant in that specific area.

Claim 7

When the inside diameter of a needle cannula is increased by washing a metal etching liquid through the needle cannula it is important to make sure that every batch of the needle cannulae exposed to the process has the same inside diameter.

One way of batch-to-batch verification is as follows. When a batch of needle cannulae is loaded in the hollow holding tool the only way a gas such as air or nitrogen can escape from the inside of the holding tool is through the lumen of individual needle cannulae. In one embodiment a gas at an overpressure (i.e. a pressure above the pressure of the surroundings) is provided at one end of the needle cannulae e.g. by applying an overpressure to the holding tool. The pressure is then monitored over a period of time. Since the gas can only escape through the needle cannulae, the decrease or drop in gas pressure over time will depend solely on the flow through the needle cannulae. Since the inside diameter of the needle cannulae is an important parameter of the flow, a mathematical equation for the inside diameter can be established. By comparing the pressure drop to a predetermined value, the internal diameter of the needle cannulae can be estimated. The test is preferably executed during the process such that the process can be continued if the inside diameter is not as wanted. If the system is not completely airtight such that gas can escape otherwise than through the lumen of the needle cannulae this can be calculated into the predetermined value set for the process.

Claim 8

In another embodiment a gas is set to flow through the needle cannulae and the flow is measured. When the gas flows through the needle cannula at a steady state, the mass flow of the gas is an expression of the inside diameter and can be compared to a predetermined value.

In both embodiments the measurement is done over a batch containing a large number of needle cannulae. The result of the test is therefore an average for the entire batch, i.e. a characterization of the batch. The presumption is therefore that the etchant works identically inside each needle cannula of the batch such that all needle cannulae of a batch has the same inside diameter. However, either of the tests can be carried out for each individual needle cannula if so wanted.

### DEFINITIONS

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. Further the term “injection needle” defines a hollow piercing member adapted to penetrate the skin of a subject for the purpose of delivering or removing a liquid. The term “Needle Cannula” is used to describe the actual conduit performing the penetration of the skin during injection. A needle cannula is usually made from a metallic material such as stainless steel and connected to a hub to form an injection needle assembly. The “hub” being the part the needle cannula is mounted to and which carries the connecting means for connecting the needle cannula to an injection apparatus is usually moulded from a suitable thermoplastic material. The “needle assembly” is to be understood as the needle unit itself i.e. comprising a needle cannula mounted in a hub as supplied to the user.

“Cartridge” is the term used to describe the container containing the drug. Cartridges are usually made from glass but could also be made from any suitable polymer e.g. by moulding or extrusion. A cartridge or ampoule is preferably sealed at one end by a pierceable membrane which can be pierced e.g. by a needle cannula. The opposite end is closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. As an alternative to a cartridge, a flexible reservoir could be used.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a cross sectional view of the needle cannula according to the invention.

FIG. 2 show a cross sectional view of the needle cannula through the line X-X on FIG. 1

FIG. 3 show a test set-up for testing the internal diameter of a needle cannula

FIG. 4 show an alternative test set-up for testing the internal diameter of a needle cannulae

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT:

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the needle cannula penetrating the patient whereas the term “proximal end” is meant to refer to the opposite end of the needle cannula.

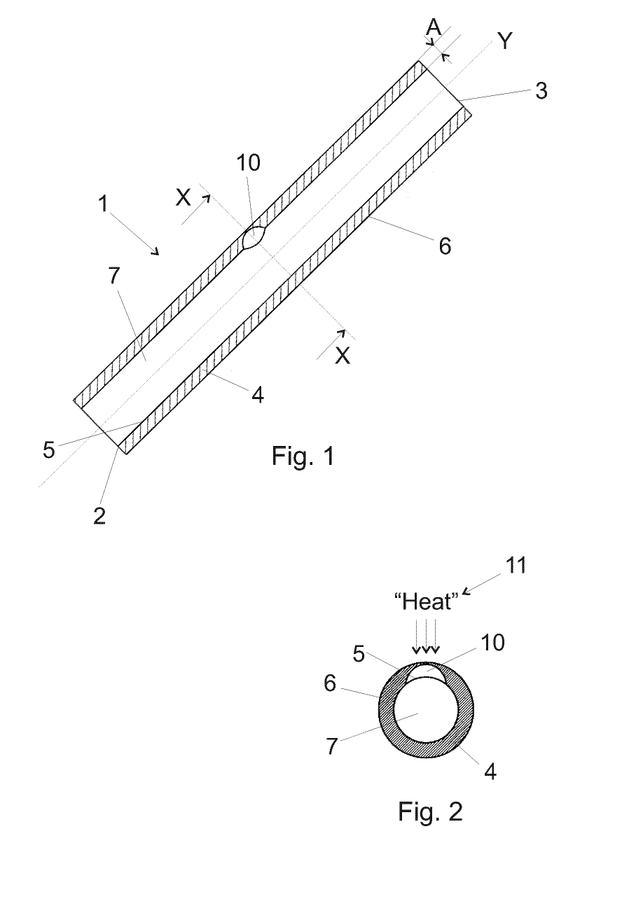

FIG. 1 discloses a needle cannula 1 having a skin piercing distal end 2 and an opposite end 3 and a sidewall 4 stretching there between. The sidewall 4 has an inside surface 5 and an outside surface 6 defining a wall thickness A. The lumen 7 through which the drug flows during injection stretches parallel to the longitudinal axis Y of the needle cannula 1.

A collapse zone 10 is formed in the inside surface 5 of the sidewall 4. The collapse zone 10 is carved out in the sidewall 4 such that the wall thickness A is diminished in the collapse zone such that the wall thickness A is locally smaller than in the remaining part of the needle cannula 1.

During the process of feeding a metal etching liquid into the lumen 7 of the needle cannula 1, the outside surface 6 is locally exposed to a heat source 11 which locally heats the sidewall 4 of the needle cannula 1 thereby making the metal etching liquid more aggressive in that particular area such that the collapse zone 10 is formed.

After, or even during, the process of feeding the metal etching liquid into the lumen of each needle cannula 1 which expands the internal diameter it would be an advantage if the expansion of the internal diameter could be verified. The metal etching liquid can either be washed all the way through the needle cannula 1 or only through a part of the length of the needle cannula 1 as disclosed in PCT/EP2010/063787. This test can be made after the process or during the process by stopping the flow of metal etching liquid for a while. The test is preferably done by passing a gas through the lumen of the needle cannulae 1. The test is not limited for needle cannulae having a collapse zone but can be utilized for any kind of needle cannulae for determination of the inside diameter. What is actually being tested is the flow through the bundle of needle cannulae, but since the average diameter through the entire length of each needle cannula is a parameter of the mathematical equation for the flow, the inside diameter of the bundle of needle cannulae is also being indirectly determined.

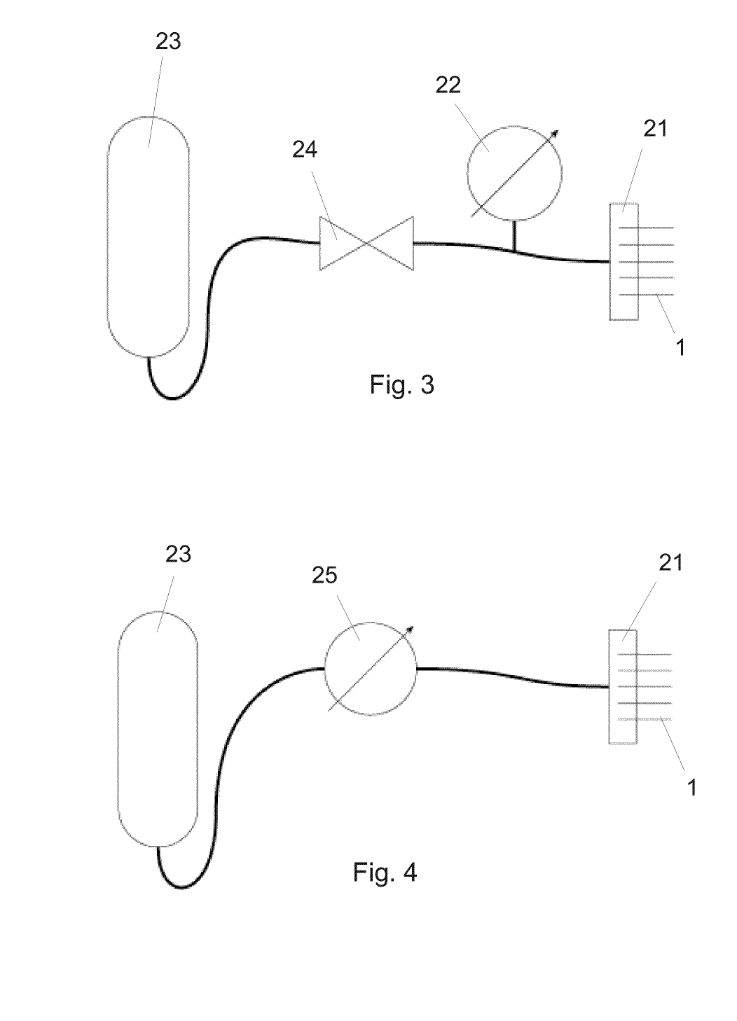

As depictured in FIG. 3, the needle container 21 holding the individual needle cannulae 1 can be filled with air from a compressor 23 with the valve 24 open until a predetermined pressure is reached. Once the decided pressure is obtained the compressor 23 is shut down and the valve 24 is closed. Now the pressurized air in the needle container 21 can only escape from the system through the lumen 7 of the individual needle cannulae 1.

A pressure transducer 22 is provided in the system which monitors the pressure decrease. This measurement of the pressure drop over time is an expression of the internal diameter of the individual needle cannulae 1 and can by being compared to a predetermined value inform whether the internal diameter has the predetermined size or whether the process should be continued. If air can escape otherwise than through the needle cannulae 1 this could be incorporated in the equation of the predetermined value.

A similar test is depictured in FIG. 4, however, here the air flows constantly through the system. By measuring the flow of air with a mass-flow meter 25 it can be determined if the wanted internal diameter of the individual needle cannulae 1 have been obtained.

Some preferred embodiments have been shown in the foregoing, but it should be stressed that the invention is not limited to these, but may be embodied in other ways within the subject matter defined in the following claims.

1. An elongated tubular needle cannula, comprising
A skin piercing distal end, an opposite proximal end and a sidewall there between, the sidewall having an outer surface and an inner surface forming a lumen through the length of the needle cannula,
Characterized in that, the inner surface of the sidewall is provided with a collapse zone.

2. An elongated tubular needle cannula according to claim 1, wherein the collapse zone comprises a locally removed portion.

3. An elongated tubular needle cannula according to claim 1, wherein a portion of the material forming the side wall has been removed thereby creating the internal collapse zone.

4. An elongated tubular needle cannula according to claim 2, wherein the removed portion is carved out in the inner surface.

5. An elongated tubular needle cannula according to claim 1, wherein the wall thickness between the inner surface and the outer surface is locally diminished at the position of the collapse Zone.

6. A method of manufacturing an elongated tubular needle cannula with a collapse zone according to claim 1 comprising:
exposing the lumen of the needle cannula to a metal etching liquid, exposing the outside surface to a heat source at a restricted area.

7. A method according to claim 6 in which the restricted area exposed to heat has the approximately same size as the collapse zone.

8. A method for in-process testing the diameter of the inside lumen of a needle cannula comprising:
providing a gas under pressure at either the distal end or the proximal end of the needle cannulae,
measuring the drop in pressure of the gas over a period of time, and
comparing the measured pressure drop to a predetermined value.

9. A method for in-process testing the diameter of the inside lumen of a needle cannula comprising:
establishing a gas flow through the lumen of the needle cannula,
measuring the mass flow rate of gas flowing through the lumen of the needle cannula, and
comparing the measured mass flow to a predetermined value.

10. An elongated tubular needle cannula according to claim 3, wherein the removed portion is carved out in the inner surface.
